(19) United States
(12) Patent Application Publication
(10) Pub. No.: US 2017/0029491 A1
(43) Pub. Date: Feb. 2, 2017

(54) ABSCOPAL EFFECT OF USPIO THERANOSIS AND IMMUNOTHERAPY FOR CANCER TREATMENT

(71) Applicant: Gerald Lee Wolf, Newton, MA (US)

(72) Inventor: Gerald Lee Wolf, Newton, MA (US)

(21) Appl. No.: 15/177,510

(22) Filed: Jun. 9, 2016

Related U.S. Application Data
(60) Provisional application No. 62/197,882, filed on Jul. 28, 2015.

(51) Int. Cl. C07K 16/18; A61K 39/39; A61K 49/22; A61K 49/18; A61K 49/00; A61K 38/19; A61K 39/00

(52) U.S. Cl. CPC C07K 16/18; A61K 38/19; A61K 39/39; A61K 39/0011; A61K 49/1818; A61K 49/0089; A61K 49/22; A61B 18/14

(57) ABSTRACT

The invention disclosed herein concerns abscopal effect of USPIO theranosis and immunotherapy using the same for cancer treatment.

### REFERENCE TO RELATED APPLICATIONS

This application claims the benefit of the filing date under 35 U.S.C. §119(e) to U.S. Provisional Application No. 62/197,882, filed on Jul. 28, 2015.

This application is also related to U.S. Provisional Application No. 61/690,005, filed on Jun. 18, 2012; U.S. Provisional Application No. 61/690,006, filed on Jun. 18, 2012; U.S. Provisional Application No. 61/742,382, filed on Aug. 9, 2012; U.S. Provisional Application No. 61/743,428, filed on Sep. 4, 2012; U.S. Provisional Application No. 61/797,757, filed on Dec. 14, 2012; and U.S. Provisional Application No. 61/779,123, filed on Mar. 13, 2013; U.S. patent application Ser. No. 13/920,694, filed on Jun. 18, 2013 (Theranosis of Macrophage-Associated Diseases with Ultrasmall Superparamagnetic Iron Oxide Nanoparticles (USPIO”); and Ser. No. 14/202,044, filed on Mar. 10, 2014 (“Theranosis of Macrophage-Associated Diseases with Ultrasmall Superparamagnetic Iron Oxide Nanoparticles (USPIO)”); U.S. Provisional Application No. 61/998,854, filed on Jul. 10, 2014 (“Companion Theranostics for Macrophage-Dependent Diseases”).

The entire contents of each of the above-referenced applications, including drawings, are incorporated herein by reference.

### BACKGROUND OF THE INVENTION

Many aggressive and most metastatic cancers are composed largely of three cell populations—bulk tumor cells, cancer stem cells, and tumor-associated macrophages (TAMs). All solid tumors are focal in nature, although they become widely disseminated upon metastasis. In addition, most successful tumors have mechanisms that protect them from immunologic attack. For example, TAMs can participate in the perpetuation of the disease by, e.g., facilitating tumor growth and immunosuppression. Specifically, TAMs can act as immunosuppressive myeloid cells, suppressing CD8⁺ T cell proliferation and secreting biochemical mediators that promote cancer cell proliferation, and eventual metastasis. Thus these aggressive cancers are usually macrophage-dependent or TAM-dependent. In general, the aggressive and metastatic propensity of cancers are related to their TAM content.

TAMs retain their phagocytic capability, and when presented with phagophyllic nanoparticles from, e.g., adjacent interstitial fluids, will internalize them and aggregate them in lysosomes.

USPIO nanoparticles extravasate from the blood, and can be endocytosed by activated macrophages such as TAMs. There, USPIO nanoparticles are aggregated in huge numbers in lysosomes of the activated macrophages, and often occupy a significant volume of the activated macrophage.

Similarly, nanotherapeutics can also be accumulated in the same TAM, a process called co-localization.

In recent years, immunotherapy has become a potential way for attacking aggressive tumors. However, success of such therapy may rest upon understanding the most efficient and effective way to generate killer lymphocytes that can attack recognized tumors wherever they are located.

There are generally four pillars to cancer therapy: surgical removal, chemotherapy, radiation therapy, and thermal ablation. Surgical therapy removes the bulk of the tumor mass, but also the source of sensitizing tumor debris that could participate in the mounting of an immunologic attack. Neither chemotherapy nor radiation therapy are especially effective against cancer stem cells, and thus have limited utility for abscopal therapy. Focal heating can cause necrosis of focal cancers if effectively delivered to some or all of a particular aggressive tumor, including the cancer stem cells. Much of the resulting cellular debris slowly finds its way to the regional lymphatics.

There remains a need for better and alternative ways to effectively treat cancer.

### SUMMARY OF THE INVENTION

One aspect of the invention provides an abscopal method of treating macrophage-dependent cancer in a subject in need thereof, the method comprising administering a cancer recognition agent (CRA) into a thermally ablated cancer of the subject, or the adjacent draining lymphatics thereof.

In certain embodiments, the cancer recognition agent is a cytokine. In certain embodiments, the cancer recognition agent is an adjuvant. In certain embodiments, the cancer recognition agent is a vaccine. In certain embodiments, the cancer recognition agent is a monoclonal antibody. In certain embodiments, the cancer recognition agent is a non-virulent infectious agent.

In any of the preceding embodiments, the CRA can be administered such that tumor debris resulting from thermal ablation and the CRA are substantially simultaneously presented to APCs (antigen presenting cells) and/or Dendritic cells locally, or within the regional lymph node chain.

In any of the preceding embodiments, the CRA can be locally administered, either intratumorally or in close proximity interstitially.

In any of the preceding embodiments, the CRA may not be heat sensitive, and is administered intra- or peri-tumorally just before enhanced hyperthermia. Alternatively, the CRA may be heat sensitive, and is given in the appropriate location after the tumor has sufficiently cooled after thermal ablation.

In any of the preceding embodiments, the CRA may be administered before, immediately after, or repeatedly after thermal ablation.

In any of the preceding embodiments, more than one CRAs may be administered. For example, different CRAs may be administered simultaneously, concurrently, or sequentially, or in rotation for repeated administration.

In any of the preceding embodiments, an immunologic response may be mounted in the subject against one or more cancers at locations other than the thermally ablated cancer.

In any of the preceding embodiments, tumor burden may be reduced in the subject after treatment.

In any of the preceding embodiments, the method may further comprise thermally ablating a second cancer of the subject, and optionally administering a second cancer recognition agent (CRA) regimen into the thermally ablated second cancer.

In any of the preceding embodiments, prior to thermal ablation, the size, shape, and/or suitability for thermal therapy of a cancer of the subject may have been determined. In certain embodiments, the size, shape, and/or suitability for thermal therapy of the cancer has been determined by USPIO-assisted imaging.

In certain embodiments, USPIO-assisted imaging may comprise: i) administering to the subject a formulation of an ultrasmall superparamagnetic iron oxide (USPIO) nanoparticle, wherein the USPIO nanoparticles have an average hydrodynamic size of about 20-50 nanometers; and optionally, administering to the subject a nanoparticulate formulation of a chemotherapeutic agent against the subject's cancer; ii) waiting a pre-determined time to allow the USPIO nanoparticles to accumulate in tumor-associated macrophages (TAMs) in said subject's cancers, to produce USPIO-enhanced TAMs in USPIO-enhanced cancers; and, iii) locating the USPIO-enhanced TAMs (e.g., with MRI or ultrasound imaging) to determine the size, shape, and/or suitability for thermal therapy of one or more said USPIO-enhanced cancers.

In any of the preceding embodiments, the cancer may be thermally ablated by raising the temperature of the cancer, comprising directing laser or HIFU (high intensity focused ultrasound) energy at USPIO-enhanced TAMs.

In any of the preceding embodiments, the cancer may be an identified USPIO-enhanced cancer, and wherein thermal ablation of the cancer comprises raising and sustaining the temperature of USPIO aggregates in TAMs of the identified USPIO-enhanced cancer. For example, elevated temperature of the USPIO aggregates may lead to necrosis and/or apoptosis of the TAMs and nearby cancer cells, thereby releasing cancer antigens.

In any of the preceding embodiments, the method may comprise repeating a step as appropriate, while tumor debris is being removed from the thermally ablated cancer by the lymphatic system.

It should be understood that any one embodiment may be combined with any one or more embodiments described herein, including those described only in the claims or the examples, unless expressly disclaimed or improper.

### DETAILED DESCRIPTION OF THE INVENTION

In general, there are two approaches for immunotherapy of cancer. In the first instance, the intent is to generate more circulating T, B, and natural killer cells that “recognize” some unique aspects of the patient's tumor that are different from non-neoplastic cells in the patient. When recognition by these effector cells, the battle proceeds and the outcome depends upon sustained efficacy in killing the tumor, wherever located. This approach might be labeled immunogenic therapy. The second approach recognizes that the problem is that the tumor killer cells that are present cannot kill the tumor because their method of attack is blocked by checkpoints on the tumor. Thus this approach advocates the use of checkpoint inhibitors to enhance immunotherapy.

A second difference in the two immunotherapy approaches is that immunogenic therapy will be elicited by local tumor interventions while checkpoint inhibitors will work following systemic administration.

In the above related applications incorporated herein by reference, the utility of USPIO formulations for imaging macrophage-dependent cancers is described. Briefly, USPIO formulations with a hydrodynamic size of 20-50 nm have massive numbers of stealth nanoparticle with long blood life. This allows them to accumulate as lysosomal aggregates in macrophage-rich cancers in a dose-dependent manner over 1-5 blood lives. The aggregates can be imaged with imaging devices such as MRI, US, or OCT, etc. The high local concentration of USPIOs facilitates absorption of any wavelength of incident laser energy, and also enhances energy deposition from focused ultrasound. Focal irradiation of the USPIO aggregates causes intense heat, which then diffuses to the adjacent malignant cancer cells. After the tumor temperature is raised to at least 47° C. and sustained for at least about 10 minutes, for example, thermal ablation follows. When the generated focal heat is higher, ablation happens more rapidly. Thus, USPIOs can be used for both diagnosis and treatment, i.e., theranosis.

Although most successful cancers are able to avoid immunodetection, the hyperthermic release of cellular contents, includes the tissue antigens of that particular cancer, renders such cellular contents available locally in high concentration, and for a sustained period. This occasionally generates a weak immunotherapeutic response.

The invention described herein provides a method to boost this (weak) immunotherapeutic response by, for example, local administration of cancer recognition agents (CRAs). The method of the invention leads to abscopal effects in the patient's system, thus treating local as well as distant tumor sites (such as secondary metastatic tumors). By effectively treating one or more of the macrophage-dependent cancers as claimed herein, abscopal responses can be obtained for immunologically similar (but distant) cancers in the patient.

According to the instant invention, in a representative embodiment, a method is provided to use nanoparticles (such as USPIOs) for imaging as well as treatment of TAM-infiltrated cancers. Focal ablation of imaged TAMs reduces the production of their immunosuppressive factors, and may account for the mild abscopal effects seen with focal therapy.

Specifically, imaging of ultrasmall superparamagnetic iron oxide particles (USPIOs) can be used to find and assess cancers having phagocytosed and accumulated USPIOs, which USPIOs can then serve as heat-enhancing agents for thermal ablation. Such USPIO nanoparticles can be imaged with any suitable imaging devices, such as MRI, ultrasound, and optical coherence tomography. The same nanoparticles can also absorb energy from a variety of energy waves impinging upon them, including laser, microwave, radiofrequency, and high intensity focused ultrasound. The local absorption of energy from one of these sources creates heat which then diffuses into the surrounding cells, including cancer cells. The killing of the macrophage and nearby cancer depends upon the degree and duration of heating or hyperthermia.

The details of hyperthermia causing either rapid necrosis or somewhat slower apoptosis are well known. There are many forms of focal therapy that can be used as part of the subject abscopal interventions for cancer treatment, and these have been demonstrated in preclinical and clinical research (see Haen, Clin. Devel. Immun. 2011:160250 (EPub 2011), incorporated by reference).

As a result of effective focal hyperthermia, the contents of the macrophage and the cancer are released into the local environment where they are slowly removed because cancers have limited lymphatics. This phenomenon, enhanced permeability and retention (EPR), is a well-known neoplastic phenomenon. When the same TAMs also contain nanotherapeutics, they will be locally released by the thermal ablation, and will tend to be retained in the poorly drained cancer. This generates enhanced local chemotherapy and synergistically generates more local tumor necrosis.

The lymphatic system serves as a conduit for macromolecules and immune cells. A large number of studies have shown that substances of nanometer size and larger are removed from the interstitium by the lymphatics. Of importance here, intravascular USPIOs identify normal and metastatic lymph nodes following intravenous injection (MEMRI). Interstitial administration of nanoprobes, including USPIOs, shows the pathway of regional lymphatics and sequential lymph node stations. The USPIO administered as described herein can identify regional nodal metastasis of the cancer, and confirms the presence of an efflux pathway for materials from a selected cancer target.

Vaccine are usually administered interstitially to target them to the regional lymphatics. Dendritic cells and APCs (antigen-presenting cells) also travel to regional lymph nodes via the peripheral lymphatics. Within the lymph node are many more dendritic cells, where, with the assistance of APCs, immuno-recognition can be initiated, resulting in generation and release of cell killing lymphocytes (T and B cells as well as NK cells).

The TAMs in aggressive cancer are responsible for stimulating local lymphangiogenesis. This can occur within the cancer, but high intratumoral pressure tends to collapse these thin-walled vessels. Thus, most of the effective lymphatics are located on the periphery of the aggressive cancer. The enhanced permeability and retention (EPR) effect in cancer emphasizes the slow lymphatic clearance of nano-sized materials from within the tumor. Importantly, following ablation, tumor debris will be slowly removed by lymphatics near the tumor. Even though the tumor cells may be rapidly killed as by thermal ablation, the tumor mass shrinks slowly due to the limited capacity of the lymphatic system to remove the debris.

The slow efflux of tumor debris and uptake by regional lymphatics provides a large repertoire of potential targets for recognition by the immune system. This includes a variety of antigens, tumor cell membrane fragments, intracellular membrane fragments, nuclear debris, and potential cytosolic immune targets. For a given tumor, more complete killing of all the cells in the tumor is likely to lead to a longer period of efflux of the tumor debris into the local lymphatics. This would increase not only temporal presentation of important immune targets, but would also allow for deeper penetration up the lymphatic chain and then into the blood stream where immunogenic responses would extend to the spleen and other sites containing APCs and dendritic cells.

This pathophysiology is beneficial for providing a sustained presentation of immune cells, tumor debris, and cancer recognition agents (CRA) to regional lymph nodes and thereby facilitates immunotherapy. Thus, interstitial administration of the CRAs into the tumor or its immediate surrounds provides optimal interactions within the lymph nodes draining the thermally ablated cancer selected for this application.

Thus one aspect of the invention provides an abscopal method of treating macrophage-dependent cancer in a subject in need thereof, the method comprising administering a cancer recognition agent (CRA) into a thermally ablated cancer of the subject, or an adjacent draining lymphatics thereof.

In certain embodiments, the method comprises administration (e.g., co-administered before, after or concurrently with thermal ablation) of cancer recognition agents (CRAs) to enhance the resulting immunotherapy, and/or to increase the abscopal effects, to effectively treat the targeted cancer, whether primary cancer or metastatic cancer.

In certain embodiments, the cancer recognition agents facilitate dendritic cell production of effective lymphocyte killer cells that recognize that particular tumor/cancer, and for the lymphocyte killer cells to circulate to some or all immunologically similar cancers of the patient.

Numerous cancer recognition agents can be used in the methods of the invention, e.g., for local administration. Such agents are chemically diverse and often have different mechanisms of action on the innate and adaptive immune system. They can be described in general classes such as adjuvants, cytokines, monoclonal antibodies, inactivated infections agents, or checkpoint inhibitors. For a given TAM-rich cancer, one or more of these agents may enhance the weak abscopal effect of focal thermal therapy.

In certain embodiments, the (locally-administered) CRA is a cytokine. CRAs in this category may include, but are not limited to: IL-2, IL-11, IL-12, GM-CSF, FLT3LG, anaphylatoxins C3a and C5a, IFN-α, IP-10, TNF-α, and IFN-γ.

In certain embodiments, the (locally-administered) CRA is an adjuvant. CRAs in this category may include, but are not limited to: aluminum salts, squalene-oil-in-water emulsions, monophosphotory lipid A, virosomes, N-dihydro-galactose-chitin (GC), saponins, CpG-oligodeoxynucleotides, GVAX, Sipuleucel-T, DepoVAX, chitin, chitosan, glycated chitosan, and polysaccharide K.

In certain embodiments, the (locally administered) CRA is a vaccine, such as Sipueucel-T (Provente), recombinant viral vaccine expressing tumor antigens, such as PSA-TRICOM, or peptide vaccine such as MAGE-A3 or NY-ES01.

In certain embodiments, the (locally-administered) CRA is a monoclonal antibody. CRAs in this category may include, but are not limited to: Ipilimumab, tremelimumab, nivolumab, MK-3475, pidilizumab, recombinant CFH, omalizumab, oregovomab, edrecolomab, TSR-042, dinutuxamab, cetuximab, avelumab, basiliximab, AGEN1884 and 2044, avelumab, pidizumab, anti-an3 integrin, and anti-GD2 3F8.

In certain embodiments, the (locally-administered) CRA may be an infectious agent that has been rendered non-virulent (e.g., a non-virulent infectious agent). CRAs in this category may include strains of mycobacteria, listeria, saccharomyces, E. coli, BCG; saccharomyces; or polio virus such as PRS-RIPO.

In certain embodiments, the cancer recognition agent is a combination of the various CRAs described herein. Thus according to the instant invention, the theranostic process previous described in the above related applications (all incorporated herein by reference) can be combined with one another, and/or with one or more checkpoint inhibitors, resulting in the immune recognition of that cancer, and the mounting of effective immunologic attack on the cancer.

The resulting immunotherapy could be effective against that cancer wherever it is located (e.g., local and/or distant). This is also known as the “abscopal effect,” or sometimes “delayed bystander effect.”

In other words, according to the method of the invention, one or more CRAs and/or one or more checkpoint inhibitors can be provided after the successful focal ablation of at least one of the patient's cancers to facilitate recognition of that cancer with the production of systemic immune response, thus effectively attacking or even eliminating (e.g., causing remission) any remaining living cancer cells that possess the recognized antigen(s). As an important result, any remaining cancer, whether still present locally or present elsewhere in the body as either primary or metastatic foci, could be eliminated or ameliorated.

In certain embodiments, the abscopal response is initiated by treating any (aggressive) tumor location containing the TAMs that are required for enhanced hyperthermia as well as companion nanotherapeutics where appropriate. It is inherent in the abscopal response that the therapy is effective on all immunologically similar cancers.

In certain embodiments, the macrophage-dependent cancer may have metastasized, but is occult at the time the treatment is initiated. Abscopal efficacy of the method of the invention is effective for these tumor foci as well.

In certain embodiments, abscopal efficacy is evaluated by tumor shrinkage observed at non-treated sites.

In certain embodiments, the CRA can be administered such that tumor debris resulting from thermal ablation and the CRA are substantially simultaneously presented to APCs (antigen presenting cells) and/or Dendritic cells locally, or within the regional lymph node chain.

In certain embodiments, the CRA is administered proximal to the tumor-draining lymphatic tree.

In certain embodiments, the CRA is locally administered, either intratumorally or in close proximity interstitially. This may ensure that the tumor recognition occurs primarily in the local lymph nodes, but may also extend further, possibly even systemically.

In certain embodiments, the CRA is not heat sensitive, and such CRA may be administered intra- or peri-tumorally just before enhanced hyperthermia.

In certain other embodiments, the CRA is heat sensitive, and can be given in the appropriate location after the tumor has sufficiently cooled after thermal ablation.

In certain embodiments, the CRA may be administered before, immediately after, or repeatedly after thermal ablation.

In certain embodiments, one or more CRA(s) is administered. The different CRAs can be administered simultaneously, concurrently, or sequentially, or in rotation for repeated administration.

In certain embodiments, the CRA is repeatedly administered into the region locally served by the proximate lymphatics, preferably as tumor debris continues to be removed from the focally treated site. The continued presentation of tumor debris may be recognized by the continual shrinkage of the treated site.

In certain embodiments, USPIO and/or imaging is repeatedly administered/carried out, which can be helpful for following the macrophage-dependent cancer sites subsequent to one or more of the interventions taught herein.

In certain embodiments, an immunologic response is mounted in the subject against one or more cancers at locations other than the thermally ablated cancer. In certain embodiments, tumor burden is reduced in the subject after treatment.

In certain embodiments, the method may further comprise thermally ablating a second cancer of the subject, and optionally administering a second cancer recognition agent (CRA) regimen into the thermally ablated second cancer.

The immunity against those cancer antigens may be long-lived, and could provide protection against that cancer for a sustained period to prevent potential relapse or even recurrence.

Optionally, prior to thermal ablation, the size, shape, and/or suitability for thermal therapy of a cancer of the subject has been determined. In certain embodiments, the size, shape, and/or suitability for thermal therapy of the cancer may have been determined by USPIO-assisted imaging.

Such USPIO-assisted imaging may, for example, comprise: i) administering to the subject a formulation of an ultrasmall superparamagnetic iron oxide (USPIO) nanoparticle, wherein the USPIO nanoparticles have an average hydrodynamic size of about 20-50 nanometers; and optionally, administering to the subject a nanoparticulate formulation of a chemotherapeutic agent against the subject's cancer; ii) waiting a pre-determined time to allow the nanoparticles to accumulate in tumor-associated macrophages (TAMs) in said subject's cancers, to produce USPIO-enhanced TAMs in USPIO-enhanced cancers; and, iii) locating the USPIO-enhanced TAMs (e.g., with MRI or ultrasound imaging) to determine the size, shape, and/or suitability for thermal therapy of one or more said USPIO-enhanced cancers.

Other USPIO-assisted imaging is described in US 2014-0249413 A1 (incorporated herein by reference).

The result of such effective USPIO-enhanced hyperthermia, with nanotherapeutics where needed, and the response to administration of the effective CRA(s) is the clearing of a part or substantially all of that cancer from that patient, i.e., personalized medicine to treat cancer.

The thermal killing of tumor cells releases tumor debris at the treated site. When such debris has nanoparticle dimensions, only the lymphatic system can remove it and aggressive tumors are known to have limited lymphatics. Thus, even though thermal killing is rapid, it takes a long time for the tumor debris to be removed—tumor shrinkage takes days to weeks to be evident. During this long interval, there is a steady efflux of the tumor debris to the local APCs and terminal lymphatics and then transport up the draining lymphatic chain before eventually reaching the blood stream. For a given tumor, more complete killing of all the cells in the tumor is likely to lead to a longer period of efflux of the tumor debris into the local lymphatics. This would increase not only temporal presentation of important immune targets, but would also allow for deeper penetration up the lymphatic chain and then into the blood stream where immunogenic responses would extend to the spleen and other sites containing APCs and dendritic cells.

The efficiency of a vaccine largely depends upon the appropriate targeting of the innate and adaptive immune systems, mainly through the prolonged delivery of antigens and immunomodulatory substances to professional antigen-presenting cells (APC) in the lymphoid environment. Adjuvants are immunological agents that modify or augment an immune response without having any specific antigenic effect of their own. Adjuvants have been developed to improve vaccination and may also be cancer recognition agents for this invention. Cancer immunotherapy has also been studied with other cancer recognition agents such as checkpoint inhibitors, specific monoclonal antibodies, therapeutic cancer vaccines, or even adoptive cell therapies. Without attempting to list all potential approaches that actively support cancer immunotherapy, the invention described herein provides useful USPIO-enhanced theranosis and abscopal therapy of macrophage-dependent cancers.

The use of USPIO formulations to identify (e.g., diagnose) and then enhance focal hyperthemic ablation of a macrophage-dependent cancer provides effective cancer theranosis as the first step. The ability to use imaging to identify the extent of macrophage-dependent cancer in a potential subject is beneficial. Critical clinical decisions may depend upon the imaging step. In certain embodiments, the method of the invention identifies, or otherwise provides information about, the location, size, and/or accessibility of any macrophage-dependent cancer focus, as well as the avidity for and distribution of the USPIOs in that cancer's TAMs. Such information can guide the development of an appropriate plan for hyperthermic ablation.

A large number of potentially useful diathermia devices may be used for the method of the invention. In certain embodiments, selection of the cancer to be treated depends upon the expected ability of the device to effectively heat the TAMs and the adjacent cancer cells in the tumor/cancer. Mild hyperthermia (e.g., 41-45° C.) is generally not expected to be sufficient to ablate the target cancer. In certain embodiments, thermal ablation useful herein is characterized by temperatures above 47° C. that are sustained for an effective period of time (e.g., 5, 10, 15, 20, 25, 30 min. or more).

Thus in certain embodiments, the cancer is thermally ablated by raising the temperature of the cancer, comprising directing laser or HIFU (high intensity focused ultrasound) energy at USPIO-enhanced TAMs.

In certain embodiments, the cancer is an identified USPIO-enhanced cancer, and wherein thermal ablation of the cancer comprises raising and sustaining the temperature of USPIO aggregates in TAMs of the identified USPIO-enhanced cancer.

In certain embodiments, the length of hyperthermic ablation depends upon the temperature generated in the TAMs. In certain embodiments, the length of hyperthermic ablation depends upon the density of USPIOs in the TAMs, which density may be approximated based on the imaging step.

In certain embodiments, during hyperthermic intervention, the generated temperatures can be monitored by simultaneous MR thermography, or by thermal probes positioned within the target, or estimated by thermal biosimulation based upon the local USPIO content.

Although the major heat sink in the selected cancer is the aggregated USPIOs, there are no barriers to heat diffusion within the cancer, and thus the absorbed heat will spread to the intimately adjacent cancer cells, rapidly killing them. Thus in certain embodiment, thermal ablation is conducted as a higher temperature, e.g., with the temperature range of about 50-99° C. (e.g., 60-89° C., 70-79° C.) and sustained in the target, for more rapid and complete cancer ablation.

Since clearance of tumor debris is slow, more ablation will lead to longer delivery of tumor debris to the local APCs. Thus, in certain embodiments, chemotherapeutics targeting the TAMs may also be used (e.g., co-administered before, after or concurrently with thermal ablation) to extend the ablation.

Several benefits accrue from the instant invention. One of the benefit of the invention is that the amount of CRA required to initiate the immunotherapy is reduced—by local as opposed to systemic administration. The reduced CRA dose may also minimize potential adverse effects of the treatment. Another benefit is that the site selected for enhanced hyperthermia can be the one most ideal (due to size, location, accessibility, stage, etc.) for any given treatment. A further benefit is that the immunotherapy is directed against all cancer cells present in the treated site, including cancer stem cells. Additionally, the CRA regimen can include multiple CRAs in combination or rotation as most effective. Finally, successful immunotherapy is achieved for that tumor and can be durable so long as the induced immunity remains. These non-limiting benefits have long been the goals of personalized medicine for patients with aggressive primary or metastatic tumors.

### REFERENCES

Ahsan, F et al., “Targeting to macrophages: role of physicochemical properties on particulate carriers—liposomes and microspheres—on the phagocytosis by macrophages,” J. Control Rel., 79:29-40 (2002).
Alexis, F et al., “Nanoparticle technologies for cancer therapy,” Handb. Esp. Pharmacol., 197:55-86 (2010).
Amoozgar, Z and Goldberg, M S, “Targeting myeloid cells using nanoparticles to improve cancer immunotherapy,” Adv. Drug Delivery Rev., 91:38-51 (2015).
Banday, A H et al., “Cancer vaccine adjuvants—recent clinical progress and future perspectives,” Immunopharmacol. Immunotoxin, 37:1-11(2015).
Baronzio, F et al., “Hyperthermia and immunity. A brief overview,” In Vivo, 20:698-695 (2008).
Bertrand, N et al., “Cancer Nanotherapeutics: the impact of passive and active targeting: the era of modern cancer biology,” Adv. Drug Delivery Rev., 66:2-26 (2014).
Brown, M C and Gromeier, M, “Oncolytic immunotherapy through tumor-specific translation and cytotoxicity of poliovirus,” Discov. Med., 19:359-65 (2015).
Bushey, R T et al., “A therapeutic antibody for cancer, derived from single human cells,” Cell Reports, 15:1-9 (2016).
Butterfield, L H, “Cancer vaccines” BMJ, 22:350 (2015).
Cherukuri, P et al., “Targeted hyperthermia using metal nanoparticles,” Adv. Drug Delivery Rev., 62:339-345 (2010).
Cheung, A S et al., “Adjuvant-loaded subcellular vesicles derived from disrupted cancer cells for cancer vaccination,” Small, EPub Mar. 8, 2016.
Christiansen, A and Detmar, M “Lymphangiogenesis and cancer,” Genes Cancer, 2:1146-1158 (2011).
den Brok, M H et al., “Efficient loading of dendritic cells following cryo and radiofrequency ablation in combination with immune modulation induces anti-tumor immunity,” Brit. J. Cancer, 95:896-905 (2005).
den Brok, M H et al., “In situ tumor ablation creates an antigen source for the generation of antitumor immunity,” Cancer Res., 64:4024-4029 (2004).
den Brok, M H et al., “Saponin-based adjuvants create a highly effective anti-tumor vaccine when combined with in situ tumor destruction,” Vaccine, 30:737-744 (2012).
Eby, J K et al., “Polymer micelles with pyridyl disulfide-coupled antigen travel through lymphatics and show enhanced cellular responses following immunization,” Acta Biomaterialia, 8:3210-3217 (2012).
Farkona, S et al., “Cancer immunotherapy: the beginning of the end of cancer?” BMC Med., 14:73-91 (2016).
Finn, O J, “Immuno-oncology: understanding the function and dysfunction of the immune system in cancer,” Ann. Oncol., Supp 8:6-9 (2012).
Frey, B et al., “Old and new facts about hyperthermia-induced modulations of the immune system,” Int. J. Hyperthermia, 28:528-542 (2012).
Goldenberg, D M and Langner, M, “Direct abscopal antitumor action of local hyperthermia,” Z. Naturforsch B., 26:359-361 (1971).
Grover, A et al., “Heat killed Saccharomyces cervisiae as an adjuvant for the induction of vaccine-mediated immunity against infection with Mycobacterium tuberculosis,” Vaccine (2016 Epub)
Gu, L and Mooney D J. “Biomaterials and emerging anticancer therapeutics: engineering the microenvironment,” Nat. Rev. Cancer, 16:56-66 (2015).
Haen, S P et al., “More than just tumor destruction: immunomodulation by thermal ablation of cancer,” Clin. Devel. Immun., 160250 (EPub (2011).
Hanson, M C et al., “Nanoparticulate STING agonists are potent lymph node-targeted vaccine adjuvants,” J. Clin. Invest., 125:2532-2546 (2015).
Hu, Z et al., “Investigation of HIFU—induced anti-tumor immunity in murine tumor model,” Transl. Med., 5:34 (2007).
Huang, X et al., “M-HIFU inhibits tumor growth, suppresses STAT3 activity and enhances tumor specific immunity in a transplant tumor model of prostate cancer” PloS One, 7:e41632 (2012).
Kalbasi, A et al., “Radiation and immunotherapy: a synergistic combination,” J. Clin. Invest., 123:2756-2763 (2013).
Khourtis, I C et al., “Peripherally administered nanoparticles target monocytic myeloid cells, secondary lymphoid organs and tumors in mice,” PLoS One, 8:e61646 (2013).
Koshy, S T and Mooney D J, “Biomaterials for enhancing anti-cancer immunity,” Curr. Opin. Biotechnol., 40:1-8 (2016).
Kourtis, I C et al., “Peripherally administered nanoparticles target monocytic myeloid cells, secondary lymphoid organs and tumors in mice,” PLoS One, 8:e61646 (2013).
Kruse, D et al., “Short-duration-focused ultrasound stimulation of Hsp 70 expression in vivo,” Phys. Med. Biol., 53:3641-3660 (2008).
Madersbacher, S et al., “Regulation of heat shock protein 27 expression of prostatic cells in response to heat treatment,” The Prostate, 37:174-181 (1998).
Manolofa, V et al., “Nanoparticles target distinct dendritic cell populations,” Eur. J. Immunol., 38:1404-1413 (2008).
Manolova, V et al., “Nanoparticles target distinct dendritic cell populations according to their size,” Eur. J. Immunol., 38:1404-1413 (2008).
McCauley, T R et al., “Pelvic lymph node visualization with MR imaging using local administration of ultra-small superparamagnetic iron oxide contrast,” J. Magn. Reson. Imaging, 15:492-497 (2002).
Muckle, D S and Dickson, J A, “Hyperthemia (42 degrees C.) as an adjunct to radiotherapy and chemotherapy in the treatment of allogeneic VX2 carcinoma in the rabbit,” Br. J. Cancer, 27:307-315 (1973).
Nishioka, Y and Yoshino, H, “Lymphatic targeting with nanoparticulate system,” Adv. Drug Delivery Rev., 42(1):55-64 (2001).
Reddy, S T et al., “In vivo targeting of dendritic cells in lymph nodes with poly (propylene sulfide) nanoparticles,” J. Control Release, 112:26-34 (2006).
Sapareto, S A and Dewey, W C, “Thermal dose determination in cancer therapy,” Int. J. RadiaL Oncol. Biol. Phys., 10:787-800 (1984).
Sharabi, A B et al., “Stereotactic radiation therapy augments antigen-specific PD-1-mediated antitumor immune responses via cross-presentation of tumor antigen,” Cancer Immunol. Res., 3:345-355 (2014).
Song, A S et al., “Thermally induced apoptosis necrosis, and heat shock protein expression in three-dimensional culture,” J. Biomech. Engin., 136(7):4027272 (2014).
Strauch, E D et al., “Combined hyperthermia and immunotherapy treatment of multiple pulmonary metastases in mice,” Surg. Oncol., 1994:45-52 (1994).
Toraya-Brown, S and Fiering, S, “Local tumor hyperthermia as immunotherapy for metastatic cancer,” Int. J. Hyperthermia, 30:531-539 (2014).
Unga J, and Hashida M, “Ultrasound induced cancer immunotherapy,” Adv. Drug Delivery Rev., 72:144-153 (2008).
Wang, C et al., “Enhanced cancer immunotherapy by microneedle patch-assisted delivery of anti-PDF antibody,” Nano. Lett., (Mar. 24, 2016 EPub).
Wang, H et al., “Abscopal antitumor immune effects of magnet-mediated hyperthermia at a high therapeutic temperature on Walker-256 carcinosarcomas in rats,” Oncol. Lett., 7:764-770 (2014).
Wu, F et al., “Expression of tumor antigens and heat-shock protein 70 in breast cancer cells after high-intensity focused ultrasound ablation,” Ann. Surg. Oncol., 14:1237-1242 (2007).
Yang, R et al., “Effects of high-intensity focused ultrasound in the treatment of experimental neuroblastoma,” J. Pediatric Surg., 27:246-250 (1992).
Zhang, H G et al., “Hyperthermia on immune regulation: a temperature's story,” Cancer Lett., 27:191-204 (2008).
US 2005/0090732 A1, Ivkov, “Therapy via targeted delivery of nanoscale particles”
US 2009/0004113, Wolf, “Macrophage-Enhanced MRI (MEMRI)”
US 2012/0003160, Wolf, “Macrophage-enhanced MRI (MEMRI) in a single imaging session”
US 2014/0249412 A1, Wolf, “Theranosis of macrophage-associated diseases with ultrasmall superparamagnetic iron oxide nanoparticles (USPIO)”
US 2015/0202291, Bosch, “Combinations of checkpoint inhibitors and therapeutics to treat cancer”
U.S. Pat. No. 5,496,536, Wolf, “Percutaneous Lymphography”
U.S. Pat. No. 8,021,689, Reddy, “Nanoparticles for immunotherapy”
WO 2013079980 A1, Akle, “Immunogenic treatment of cancer”
WO 2016007194 A1, Wolf, “Theranostics for macrophage-dependent diseases”

All references cited herein are incorporated by reference.

1. An abscopal method of treating macrophage-dependent cancer in a subject in need thereof, the method comprising administering a cancer recognition agent (CRA) into a thermally ablated cancer of the subject, or an adjacent draining lymphatics thereof.

2. The method of claim 1, wherein the cancer recognition agent is a cytokine, an adjuvant, a vaccine, a monoclonal antibody, or a non-virulent infectious agent.

3-6. (canceled)

7. The method of claim 1, wherein the CRA is administered such that tumor debris resulting from thermal ablation and the CRA are substantially simultaneously presented to APCs (antigen presenting cells) and/or Dendritic cells locally, or within the regional lymph node chain.

8. The method of claim 1, wherein the CRA is locally administered, either intratumorally or in close proximity interstitially.

9. The method of claim 2, wherein the CRA is not heat sensitive, and is administered intra- or peri-tumorally just before enhanced hyperthermia.

10. The method of claim 2, wherein the CRA is heat sensitive, and is given in the appropriate location after the tumor has sufficiently cooled after thermal ablation.

11. The method of claim 1, wherein the CRA is administered before, immediately after, or repeatedly after thermal ablation.

12. The method of claim 1, wherein more than one CRAs is administered.

13. The method of claim 12, wherein different CRAs are administered simultaneously, concurrently, or sequentially, or in rotation for repeated administration.

14. The method of claim 1, wherein an immunologic response is mounted in the subject against one or more cancers at locations other than the thermally ablated cancer.

15. The method of claim 1, wherein tumor burden is reduced in the subject after treatment.

16. The method of claim 1, further comprising thermally ablating a second cancer of the subject, and optionally administering a second cancer recognition agent (CRA) regimen into the thermally ablated second cancer.

17. The method of claim 1, wherein prior to thermal ablation, the size, shape, and/or suitability for thermal therapy of a cancer of the subject has been determined.

18. The method of claim 17, wherein the size, shape, and/or suitability for thermal therapy of the cancer has been determined by USPIO-assisted imaging.

19. The method of claim 18, wherein USPIO-assisted imaging comprises:
i) administering to the subject a formulation of an ultrasmall superparamagnetic iron oxide (USPIO) nanoparticle, wherein the USPIO nanoparticles have an average hydrodynamic size of about 20-50 nanometers; and optionally, administering to the subject a nanoparticulate formulation of a chemotherapeutic agent against the subject's cancer;
ii) waiting a pre-determined time to allow the USPIO nanoparticles to accumulate in tumor-associated macrophages (TAMs) in said subject's cancers, to produce USPIO-enhanced TAMs in USPIO-enhanced cancers; and,
iii) locating the USPIO-enhanced TAMs (e.g., with MRI or ultrasound imaging) to determine the size, shape, and/or suitability for thermal therapy of one or more said USPIO-enhanced cancers.

20. The method of claim 1, wherein the cancer is thermally ablated by raising the temperature of the cancer, comprising directing laser or HIFU (high intensity focused ultrasound) energy at USPIO-enhanced TAMs.

21. The method of claim 1, wherein the cancer is an identified USPIO-enhanced cancer, and wherein thermal ablation of the cancer comprises raising and sustaining the temperature of USPIO aggregates in TAMs of the identified USPIO-enhanced cancer.

22. The method of claim 21, wherein elevated temperature of the USPIO aggregates lead to necrosis and/or apoptosis of the TAMs and nearby cancer cells, thereby releasing cancer antigens.

23. The method of claim 1, comprising repeating a step as appropriate, while tumor debris is being removed from the thermally ablated cancer by the lymphatic system.
